(19) United States
(12) Patent Application Publication
(10) Pub. No.: US 2016/0113684 A1
(43) Pub. Date: Apr. 28, 2016

(54) SPINAL IMPLANT SYSTEM AND METHODS OF USE

(71) Applicant: Warsaw Orthopedic, Inc., Warsaw, IN (US)

(72) Inventors: William Alan Rezach, Atoka, TN (US); Jason May, Cordova, TN (US); Nicholas Benson, Cordova, TN (US)

(21) Appl. No.: 14/522,321

(22) Filed: Oct. 23, 2014

(51) Int. Cl. A61B 17/70; A61B 17/86

(52) U.S. Cl. CPC A61B 17/7032; A61B 17/7037; A61B 17/863; A61B 2017/00526

(57) ABSTRACT

A spinal implant system comprises at least one first member including an inner surface defining an implant cavity. A second member is configured to penetrate tissue and includes an inner surface defining a cavity. A third member is engageable with the inner surfaces to resist and/or prevent movement of the second member relative to the first member. Fasteners, instruments and methods are disclosed.

### TECHNICAL FIELD

The present disclosure generally relates to medical devices for the treatment of spinal disorders, and more particularly to a surgical implant system including a bone fastener.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, spinal constructs such as vertebral rods are often used to provide stability to a treated region. Rods redirect stresses away from a damaged or defective region while healing takes place to restore proper alignment and generally support the vertebral members. During surgical treatment, one or more rods and bone fasteners can be delivered to a surgical site. The rods may be attached via the fasteners to the exterior of two or more vertebral members. This disclosure describes an improvement over these prior art technologies.

### SUMMARY

In one embodiment, a spinal implant system is provided. The spinal implant system comprises at least one first member including an inner surface defining an implant cavity. A second member is configured to penetrate tissue and includes an inner surface defining a cavity. A third member is engageable with the inner surfaces to resist and/or prevent movement of the second member relative to the first member. In some embodiments, fasteners, instruments and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a spinal implant system in accordance with the principles of the present disclosure;

FIG. 2 is a perspective view of components shown in FIG. 1;

FIG. 3 is a perspective view, with parts separated, of the components shown in FIG. 2;

FIG. 4 is a cross section view of the components shown in FIG. 2;

FIG. 4A is a perspective view of a component shown in FIG. 2;

FIG. 5 is a side view of a component shown in FIG. 1;

FIG. 6 is a break away view of the component shown in FIG. 5; and

FIG. 7 is a cross section view of the components shown in FIG. 1.

### DETAILED DESCRIPTION

The exemplary embodiments of a surgical system and related methods of use disclosed are discussed in terms of medical devices for the treatment of musculoskeletal disorders and more particularly, in terms of a spinal implant system including a bone fastener. In one embodiment, the spinal implant system includes an implant comprising a bone fastener, such as, for example, a universal pedicle bone screw.

In one embodiment, the spinal implant system comprises a modular system including an array of members, such as, for example, receivers that can be selectively coupled to members, such as, for example, bone screw shafts. In some embodiments, the spinal implant system provides bone screw options while providing fixed axis screw (FAS) functionality.

In some embodiments, the spinal implant system includes a crown configured to mate with a drive portion of a head of a screw shaft. In some embodiments, the crown is configured to prevent the shaft from rotating along any axis and/or within any plane. In some embodiments, the crown is employed with a receiver having uniaxial screw (UAS) functionality. In some embodiments, the crown is employed with a receiver having multi-axial screw (MAS) functionality. In one embodiment, the crown is employed with a receiver having UAS functionality to withstand derotation forces such that the load from the derotation forces are dispersed between a bone screw shaft and a receiver interface, and a bone screw shaft and a crown interface. In one embodiment, the spinal implant system includes a two piece crown.

In one embodiment, the spinal implant system comprises a bone fastener having a universal screw design that includes two flats and/or keyed geometry to facilitate FAS functionality. In one embodiment, the spinal implant system comprises a bone fastener having a universal screw design that includes a spherical head to facilitate FAS functionality. In some embodiments, the spinal implant system includes a bone fastener having a band, such as, for example, a ring that has a gap smaller than its height and/or width. In some embodiments, this configuration maintains strength performance of the spinal implant system. In some embodiments, the spinal implant system is employed with a method of selectively mating members, such as, for example, heads and shafts, of the spinal implant system to achieve FAS functionality, UAS functionality or MAS functionality from the same bone fastener depending on the selected head being placed onto the shaft.

In some embodiments, the spinal implant system includes a bone fastener having a screw shank. In some embodiments, the screw shank includes a mating element, such as, for example, flats on an outside surface thereof. In some embodiments, the bone fastener includes a ring disposed with a receiver connected with the screw shank. In some embodiments, the ring has a minimal gap to increase a retaining strength of the screw shank. In some embodiments, the ring has a gap, such as, for example, a slot having a slot thickness less than the height or width of the ring.

In some embodiments, the spinal implant system is employed with a method that includes the step of inserting the ring with the head. In some embodiments, the method includes the step of assembling a head with a shaft to achieve FAS functionality. In some embodiments, the method includes the step of assembling a head with a shaft to achieve FAS functionality such that the crown engages an opening in the head.

In some embodiments, the spinal implant system includes a bone fastener that includes a shaft having at least one flat for keying into a receiver. In some embodiments, the bone fastener includes a ring having a cylindrical shape. In some embodiments, the method includes the step of assembling a bone fastener by employing a flexible wire insertion technique with the ring. In some embodiments, the method includes the step of inserting the ring into the head and through a hole of the head and then the ring deforms into a C type of arrangement. In some embodiments, the method includes the step of attaching modular heads that provide different degrees of freedom to the same bone screw shaft depending on what head is placed onto the bone screw shaft. In some embodiments, the spinal implant system comprises a modular kit that includes a universal screw shank and modular heads that can interface differently, for example, in a multi-axial, uni-axial and/or fixed axis configuration.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed spinal implant system may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic, sacral and pelvic regions of a spinal column. The spinal implant system of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. As used in the specification and including the appended claims, the term “tissue” includes soft tissue, ligaments, tendons, cartilage and/or bone unless specifically referred to otherwise.

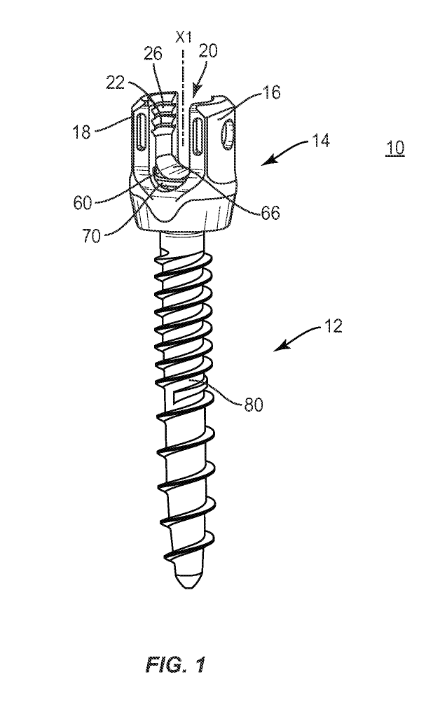

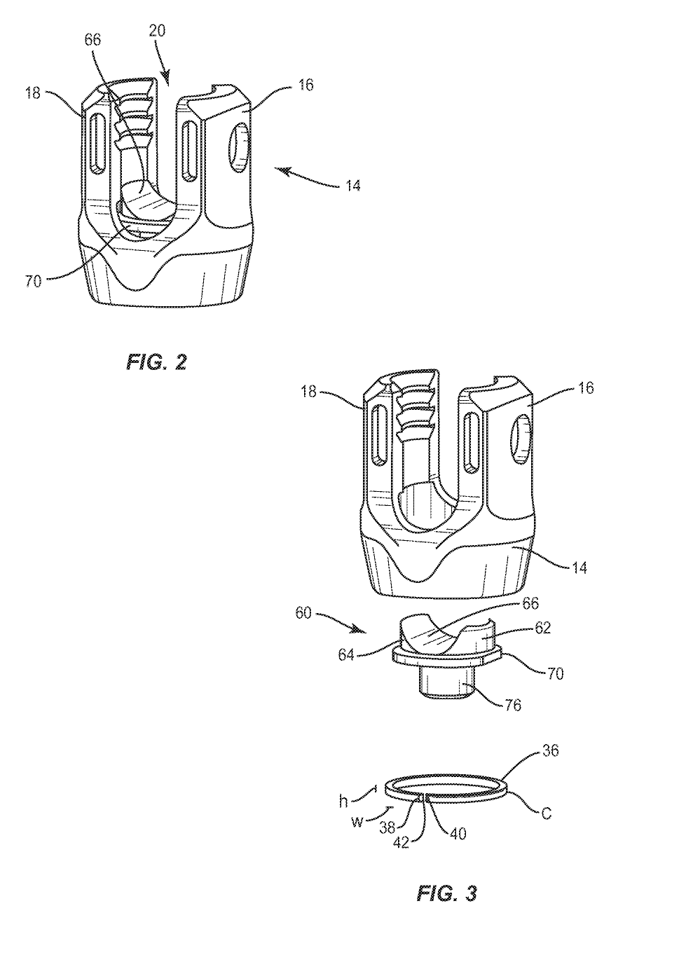

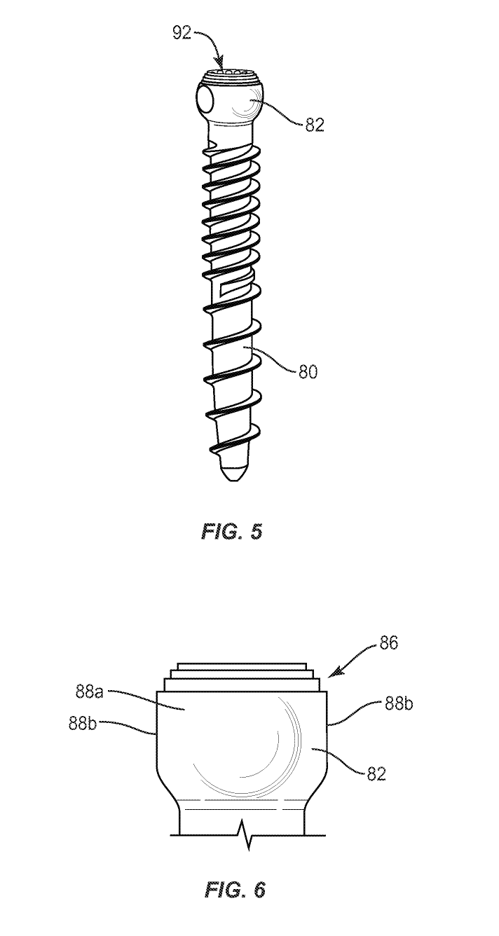

The following discussion includes a description of a surgical system including a bone fastener, related components and methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-7, there are illustrated components of a spinal implant system 10 including a plurality of alternate bone fastener configurations, such as, for example, a plurality of bone screw configurations 12.

The components of spinal implant system 10 can be fabricated from biologically acceptable materials suitable for medical applications, including metals, synthetic polymers, ceramics and bone material and/or their composites. For example, the components of spinal implant system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TOP), hydroxyapatite (HA)-TCP, calcium sulfate, or other resorbable polymers such as polyaetide, polyglycolide, polytyrosine carbonate, polycaroplaetohe and their combinations.

Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening dements and/or instruments, as described herein.

In some embodiments, spinal implant system 10 comprises a spinal implant kit, which includes a plurality of members, such as, for example, implant receivers 14. Each receiver 14 is configured for selection from the plurality of receivers such that receiver 14 is connectable with an interchangeable member, such as, for example, a shaft 80. An interchangeable mating dement, such as, for example, a head 82 of shaft 80 is engageable with a mating dement, as described herein, of a selected receiver 14 for selected movement of its components parts and/or movement relative to tissue to form a selected bone screw 12. In some embodiments, receiver 14 is configured for selection from the plurality of receivers such that receiver 14 is connectable with a compatible shaft 80.

In some embodiments, shaft 80 is connected to a selected receiver 14 to comprise a fixed axis fastener to resist and/or prevent movement of shaft 80 relative to receiver 14. In some embodiments, receiver 14 comprises a multi-axial receiver. In some embodiments, receiver 14 comprises a uni-axial receiver. In some embodiments, receiver 14 comprises one or more fixed axis receivers, multi-axial receivers and uni-axial receivers. In some embodiments, spinal implant system 10 comprises a spinal implant kit, which includes receivers 14 and alternate receivers, such as those described herein.

Each receiver 14 extends along and defines an axis X1. Receiver 14 includes a pair of spaced apart arms 16, 18 that define an implant cavity 20 therebetween configured for disposal of a component of a spinal construct, such as, for example, a spinal rod (not shown). Arms 16, 18 each extend parallel to axis X1. In some embodiments, arm 16 and/or arm 18 may be disposed at alternate orientations, relative to axis X1, such as, for example, transverse, perpendicular and/or other angular orientations such as acute or obtuse, coaxial and/or may be offset or staggered. Arms 16, 18 each include an arcuate outer surface extending between a pair of side surfaces. At least one of the outer surfaces and the side surfaces of arms 16, 18 have at least one recess or cavity therein configured to receive an insertion tool, compression instrument and/or instruments for inserting and tensioning bone screw 12.

Cavity 20 is substantially U-shaped. In some embodiments, all or only a portion of cavity 20 may have alternate cross section configurations, such as, for example, V-shaped, W-shaped, oval, oblong triangular, square, polygonal, irregular, uniform, non-uniform, offset, staggered, and/or tapered. Each receiver 14 includes an inner surface 22. A portion of surface 22 includes a thread form 24 located adjacent arm 16 and a thread form 26 located adjacent arm 18. Thread forms 24, 26 are configured for engagement with a coupling member, such as, for example, a setscrew (not shown), to retain a spinal construct, such as, for example, a spinal rod within cavity 20. In some embodiments, surface 22 may be disposed with the coupling member in alternate fixation configurations, such as, for example, friction fit, pressure fit, locking protrusion/recess, locking keyway and/or adhesive. In some embodiments, all or only a portion of surface 22 may have alternate surface configurations to enhance engagement with the spinal rod and/or the setscrew such as, for example, rough, arcuate, undulating, mesh, porous, semi-porous, dimpled and/or textured.

Each surface 22 defines a cavity, such as, for example, a groove 34 configured for disposal of a band, such as, for example, a C-shaped ring 36. Ring 36 includes a height h and a width w. Ring 36 includes a circumference C that extends between end 38 and end 40. Ends 38, 40 define an opening, such as, for example a gap 42. Gap 42 is sized such that gap 42 has a thickness that is less than height h and width w. Ring 36 is configured to engage an outer surface of head 82 and is disposable with groove 34 to prevent axial translation of shaft 80 relative to the selected receiver 14.

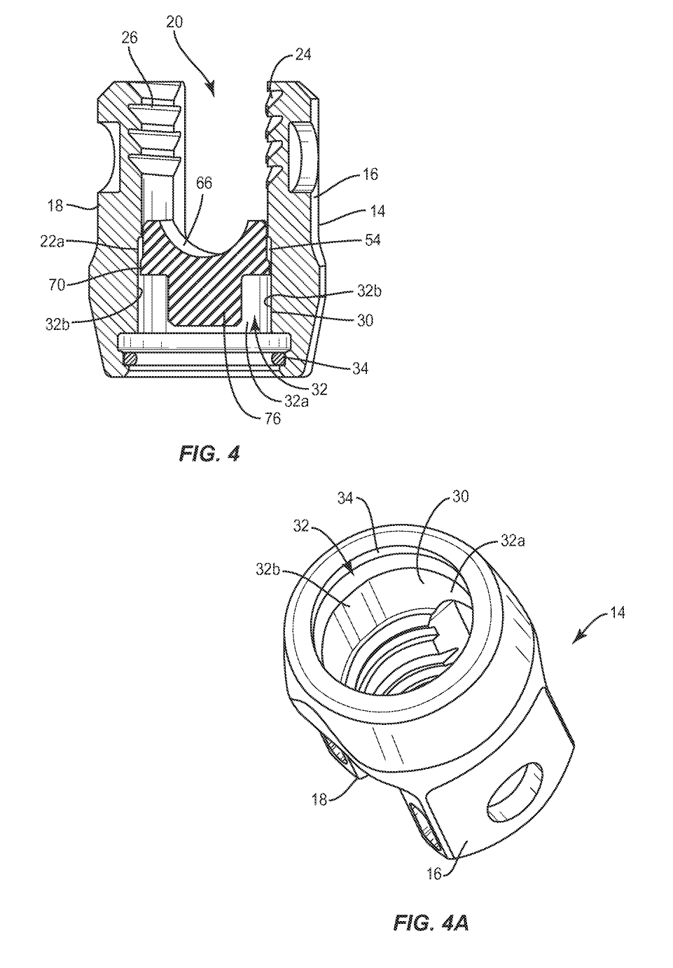

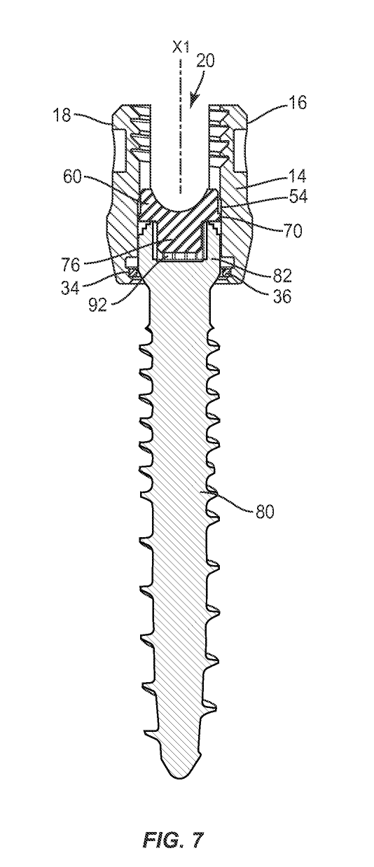

A portion of surface 22 of each receiver 14 defines a particularly configured mating element, such as, for example, an engagement surface 30 configured to interface in a selective mating engagement with head 82 of shaft 80, as shown in FIG. 7. Surface 30 includes a keyway 32 that includes mating elements, such as, for example, arcuate surfaces 32a and planar surfaces, such as, for example, flats 32b, as shown in FIGS. 4 and 4A.

Flats 32b are configured to interface with flats 88b of head 82 and arcuate surfaces 32a are configured to interface with arcuate surfaces 88a in a keyed connection, as shown in FIG. 7. Flats 32b engage flats 88b to resist and/or prevent rotation of receiver 14 about axis X1. In this configuration, shaft 80 is free to rotate along a single axis and/or within a single plane relative to receiver 14, similar to a fastener having UAS functionality. To resist and/or prevent movement of shaft 80 relative to receiver 14 along any axis or within any plane, a member, such as, for example, a crown 60 is positioned with receiver 14 and shaft 80, as described herein.

Surface 22 includes surface 22a that defines a cavity, such as, for example, a slot 54 configured for disposal of crown 60, as discussed herein. Slot 54 communicates with cavity 20. Crown 60 is configured for disposal within slot 54 of the selected receiver 14. Crown 60 includes a portion, such as, for example, a wall 62 that includes a surface 64. Surface 64 defines a concave portion, such as, for example, a saddle 66 that is disposed in cavity 20 and configured to receive a spinal implant, such as, for example, a spinal rod.

Crown 60 includes a medial portion, such as, for example, a circumferential flange 70 configured for engagement with surface 22a. Flange 70 is disposed between saddle 66 and a projection 76, as described herein. Flange 70 engages surface 22a in an abutting orientation to resist and/or prevent movement of shaft 80 relative to receiver 14, as described herein.

Crown 60 includes a portion, such as, for example, a projection 76 that engages head 82. Projection 76 is configured for mating engagement with a socket 92 of head 82. In some embodiments, projection 76 includes a hexalobe drive to mate with a corresponding configuration of socket 92, as described herein. In some embodiments, projection 76 may engage socket 92 in alternate mating configurations, such as, for example, friction fit, pressure fit, locking protrusion/recess, locking keyway and/or adhesive. In some embodiments, socket 92 has a cruciform, phillips, square, hexagonal, polygonal, star cross sectional configuration configured for disposal of a correspondingly shaped portion of a driver.

Projection 76 extends in a direction opposite from saddle 66. In some embodiments, saddle 66, flange 70 and projection 76 are monolithically formed to form crown 60. In some embodiments, crown 60 may include a plurality of components that are integrally connected or separately attachable, for example, a two piece crown.

Crown 60 is positioned in cavity 20 such that projection 76 is disposed with socket 92 in a mating engagement. Saddle 66 is disposed in cavity 20 for receiving a spinal rod and flange 70 is disposed with slot 54 to abut surface 22a and head 82. Disposal of crown 60 with cavity 20 in engagement with the surfaces of receiver 14 and shaft 80, as described herein, resists and/or prevents movement of shaft 80 relative to receiver 14 along any axis or within any plane to provide bone screw 12 with FAS functionality. As such, interchangeable head 82 is connected with surface 30 of a selected receiver 14 from the kit of receivers 14 to form a fixed axis bone screw 12. In some embodiments, engagement of crown 60 with the surfaces of receiver 14 and shaft 80 due to forces and/or stress applied to receiver 14 and/or shaft 80, such as, for example, via movement of spinal construct components and/or vertebrae, resists and/or prevents movement of receiver 14 relative to shaft 80.

In some embodiments, shaft 80 is configured to penetrate tissue, such as, for example, vertebrae. Head 82 is engageable with any of the plurality of receivers 14. Head 82 includes a substantially spherical proximal portion configured for disposal with a selected receiver 14 and engagement with crown 60. Head 82 includes a plurality of ridges 86 to improve purchase of head 82 with crown 60.

In assembly, operation and use, spinal implant system 10, similar to the systems and methods described herein, includes a selected bone screw 12, which comprises a selected receiver 14 for connection with interchangeable shaft 80 having a selected movement, and is employed with a surgical procedure for treatment of a spinal disorder affecting a section of a spine of a patient, as discussed herein. Spinal implant system 10 is employed with a surgical procedure for treatment of a condition or injury of an affected section of the spine. In some embodiments, a selected bone screw 12 comprises a selected receiver 14 for connection with a compatible shaft 80.

The components of spinal implant system 10 include a spinal implant kit, which comprises the plurality of receivers and interchangeable shafts 80. In some embodiments, spinal implant system 10 includes a spinal implant kit, which comprises the plurality of receivers and compatible shafts 80. The plurality of receivers include receivers 14 and alternate receivers, such as those described herein, that interface with interchangeable shafts 80 to comprise one or more bone screw configurations. Selected bone screws 12 and one or a plurality of spinal implants, such as, for example, vertebral rods can be delivered or implanted as a pre-assembled device or can be assembled in situ. The components of spinal implant system 10 may be may be completely or partially revised, removed or replaced.

In one embodiment, a receiver 14 is selected from the kit of the plurality of receivers 14 for connection with interchangeable shaft 80 to comprise a bone screw 12 having a selected fixed axis orientation and/or FAS functionality. Ring 36 is disposed with head 82, as described herein, such that circumference C extends about head 82 and ends 38, 40 include gap 42. Crown 60 is disposed with cavity 20 in engagement with the surfaces of receiver 14 and shaft 80, as described herein, to resist and/or prevent movement of shaft 80 relative to receiver 14 along any axis or within any plane to provide bone screw 12 with FAS functionality. In some embodiments, a selected bone screw 12 comprises a selected receiver 14 for connection with a compatible shaft 80.

In use, for treatment of a spinal disorder, shaft 80 can be threaded and engaged with tissue. In some embodiments, the selected bone screw 12 is disposed adjacent vertebrae at a surgical site and is manipulated to drive, torque, insert or otherwise connect bone screw 12 with vertebrae.

In some embodiments, spinal implant system 10 includes an agent, which may be disposed, packed, coated or layered within, on or about the components and/or surfaces of spinal implant system 10. In some embodiments, the agent may include bone growth promoting material, such as, for example, bone graft to enhance fixation of the fixation elements with vertebrae. In some embodiments, the agent may include one or a plurality of therapeutic agents and/or pharmacological agents for release, including sustained release, to treat, for example, pain, inflammation and degeneration.

In some embodiments, the use of microsurgical and image guided technologies may be employed to access, view and repair spinal deterioration or damage, with the aid of spinal implant system 10. The components of spinal implant system 10 can be made of radiolucent materials such as polymers. Radiomarkers may be included for identification under x-ray, fluoroscopy, CT or other imaging techniques.

In some embodiments, spinal implant system 10 can include one or a plurality of bone screws 12 such as those described herein and/or fixation elements, which may be employed with a single vertebral level or a plurality of vertebral levels. In some embodiments, bone screws 12 may be engaged with vertebrae in various orientations, such as, for example, series, parallel, offset, staggered and/or alternate vertebral levels. In some embodiments, bone screws 12 may be configured as multi-axial screws, sagittal angulation screws, pedicle screws, mono-axial screws, uni-planar screws, fixed screws, anchors, tissue penetrating screws, conventional screws, expanding screws. In some embodiments, bone screws 12 may be employed with wedges, anchors, buttons, clips, snaps, friction fittings, compressive fittings, expanding rivets, staples, nails, adhesives, posts, connectors, fixation plates and/or posts.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

1. A spinal implant system comprising:
at least one first member including an inner surface defining an implant cavity;
a second member configured to penetrate tissue and including an inner surface defining a cavity; and
a third member engageable with the inner surfaces to resist and/or prevent movement of the second member about one or a plurality of axes relative to the first member,
wherein the cavity of the second member comprises a hexalobe drive socket and the third member includes a hexalobe drive engageable with the inner surface defining the hexalobe drive socket.

2. (canceled)

3. A spinal implant system as recited in claim 1, wherein the third member includes a first portion disposable with the implant cavity and a second portion disposable with the cavity of the second member.

4. A spinal implant system as recited in claim 1, wherein the third member includes a concave portion engageable with a spinal implant in the implant cavity and a projection engageable with the inner surface of the second member in the cavity.

5. A spinal implant system as recited in claim 1, wherein the third member includes a crown having a saddle engageable with a spinal implant in the implant cavity and a projection engageable with the inner surface of the second member in the cavity.

6. A spinal implant system as recited in claim 1, wherein the third member includes a flange engageable with the inner surface of the first member.

7. A spinal implant system as recited in claim 1, wherein the third member includes a medial circumferential flange engageable with the inner surface of the first member.

8. A spinal implant system as recited in claim 1, wherein the cavity of the second member comprises a drive socket.

9. (canceled)

10. A spinal implant system as recited in claim 1, wherein the at least one first member comprises a plurality of alternate first members such that the second member is interchangeable with the plurality of first members.

11. A spinal implant system as recited in claim 1, wherein the first, second and third members comprise a fixed axis bone fastener.

12. A spinal implant system as recited in claim 1, wherein the at least one first member comprises at least a multi-axial bone fastener receiver.

13. A spinal implant system as recited in claim 1, wherein the at least one first member comprises at least a uni-axial bone fastener receiver.

14. A spinal implant system as recited in claim 1, wherein the second member comprises a head having the inner surface and a shaft configured to penetrate the tissue, the third member being engageable with the inner surfaces to resist and/or prevent rotation of the shaft relative to the first member about one or a plurality of axes.

15. A spinal implant system as recited in claim 1, wherein the second member comprises a head having the inner surface and a shaft configured to penetrate the tissue, the third member being engageable with the inner surfaces to resist and/or prevent pivoting of the shaft through a plurality of planes relative to the first member.

16. A spinal implant system as recited in claim 1, wherein the second member comprises a head having the inner surface and a shaft configured to penetrate the tissue, the head including flats engageable with the inner surface of the first member to resist and/or prevent rotation of the head relative to the first member.

17. A spinal implant system as recited in claim 1, wherein the inner surface of the first member defines a groove configured for disposal of a circumferential ring that defines a gap.

18. A spinal implant system as recited in claim 17, wherein the gap defines a slot thickness that is less than a height and a width of the ring.

19. A method of assembly for a bone fastener, the method comprising the steps of:
providing a plurality of alternate first members, each of the first members including an inner surface defining an implant cavity;
providing a second member being interchangeable with the plurality of first members and configured to penetrate tissue, the second member including an inner surface defining a cavity;
selecting a first member and connecting the selected first member with the second member; and
engaging a third member with the inner surfaces to resist and/or prevent movement of the second member about one or a plurality of axes relative to the first member such that the first, second and third members comprise a fixed axis bone fastener,
wherein the cavity of the second member comprises a hexalobe drive socket and the third member includes a hexalobe drive engageable with the inner surface defining the hexalobe drive socket.

20. A spinal implant system comprising:
a plurality of alternate implant receivers each including an inner surface defining an implant cavity;
a bone screw shaft including a head engageable with an implant receiver such that the shaft is compatible with the plurality of implant receivers, the head comprising an inner surface defining a cavity; and
a crown engageable with the inner surface of a selected implant receiver and the inner surface of the head to resist and/or prevent movement of the shaft about one or a plurality of axes relative to the selected implant receiver such that the selected implant receiver, the shaft and the crown comprise a fixed axis bone fastener,
wherein the cavity of the shaft comprises a hexalobe drive socket and the crown includes a hexalobe drive engageable with the inner surface defining the hexalobe drive socket.

21. A spinal implant system as recited in claim 1, wherein:
the second member comprises a head that includes the inner surface that defines the cavity; and
the third member comprises a circumferential flange that engages an uppermost surface of the head when the hexalobe drive engages the hexalobe drive socket.

22. A spinal implant system as recited in claim 1, wherein the inner surface defining the implant cavity comprises a pair of flats that engage flats of the second member when the hexalobe drive engages the hexalobe drive socket.
